(19) United States
(12) Patent Application Publication
(10) Pub. No.: US 2013/0131756 A1
(43) Pub. Date: May 23, 2013

(54) Posterior Tibial-Nerve and/or Other Nerve Stimulation System and Method

(71) Applicants: Giancarlo Barolat, Golden, CO (US); Scott F. Drees, Dallas, TX (US)

(72) Inventors: Giancarlo Barolat, Golden, CO (US); Scott F. Drees, Dallas, TX (US)

(21) Appl. No.: 13/747,293

(22) Filed: Jan. 22, 2013

Related U.S. Application Data
(63) Continuation of application No. 12/851,882, filed on Aug. 6, 2010, now abandoned.
(60) Provisional application No. 61/231,837, filed on Aug. 6, 2009.

(51) Int. Cl. A61N 1/36

(52) U.S. Cl. CPC A61N 1/36071
USPC 607/46

(57) ABSTRACT

An implantable pulse generator includes one or more structural features for accommodating the shape of a portion of a patient's limb, such as the shape of the patient's calf. In one embodiment, an implantable pulse generator includes a first node interconnected to a second node by an elongated housing member, the elongated housing member including a convex surface substantially matching a curvature of the patient's limb, such as a portion of the patient's arm or leg. Alternatively, an articulating housing is associated with the implantable pulse generator for enabling a surgeon to bend the housing to substantially conform to the patient's limb, such as a portion of the patient's arm or leg.

### RELATED APPLICATION

This application claims the benefit of U.S. Provisional Patent Application No. 61/231,837 filed on Aug. 6, 2009, the content of which is expressly incorporated herein by reference.

### FIELD

The present invention is related to medical implants, and more particularly, to electrical stimulation of nerves within a patient's extremities, for the treatment of intractable pain.

### BACKGROUND

Intractable pain in a patient's limb, such as pain in the plantar surface of the foot, can be secondary to several conditions: plantar fasciitis, bunions, entrapped nerves, neuromas, nerve damage, diabetic neuropathy, to name a few. Millions of individuals in the USA suffer severe plantar foot pain which does not respond to conventional treatment modalities (medications, injections, surgical procedures, etc.).

A heel neuroma is basically a nerve entrapment that occurs amongst the inflammation that occurs in heel pain. Most patients will present with a complaint of heel pain on the bottom of the heel. After the interview, examination, x-rays and any other tests, the doctor may perform a diagnosis of plantar fasciitis or heel spur. There are other less common conditions that may also cause heel pain. It has been noticed that, in a substantial percentage of individuals where the diagnosis of plantar fasciitis or heel spur is made, there is also involvement of the “calcaneal nerve” which is a small branch that comes off the larger nerve. This nerve is located on the inside part of the heel and travels underneath the heel. The nerve can be traumatized from the constant pounding that the heel takes both in walking and exercise. This condition can also be accelerated by a degeneration of the fat pad on the bottom of the heel. In those cases where the heel pain gets worse as the patient ambulates, a diagnosis of heel neuroma has to be considered.

Interdigital neuroma, more commonly known as Mortons neuroma or “foot neuroma,” is an entrapment of a nerve that passes into the toes of the foot. The nerve becomes entrapped by the metatarsal bones. Typically this type of pain in the ball of the foot is the result of wearing shoes that are too narrow or with very flimsy soles, or in the case of women, high heel shoes. Other factors that will cause this condition can be the specific architecture of a person's foot structure, as well as occupational hazards such as those who work low to the ground forcing them to kneel down for long periods of time.

Complex Regional Pain Syndrome can be very painful. Typically, a person will be walking in shoes (although the condition can be painful out of shoes as well), and will develop a sharp pain just behind the third and fourth toes. Complex Regional Pain Syndrome is a still poorly understood neurological phenomenon that carries a number of names: Reflex Sympathetic Dystrophy (RSD), Sudecks Atrophy and causalgia. The condition is basically a short circuit of the nervous system where nerves misfire, thus sending constant pain signals to the brain. In other words, it is a completely abnormal response by the nervous system to normal external stimuli.

Metatarsal joint pain commonly results from misalignment of the joint surfaces, which puts pressure on the joint lining and destroys cartilage in the joints. Mild heat and swelling may develop pain in the ball of the foot (called metatarsalgia) and may have many different causes (including arthritis, poor circulation, pinching of the nerves between the toes, posture problems, and various disorders). However, most often the pain is caused by nerve damage or by an abnormality of the joints nearest the balls of the feet (metatarsal joints). Fat tissue, which helps cushion the joints when bearing weight can be pushed forward under the toes, resulting in a loss of cushioning. This loss of cushioning can also damage the nerves in the ball of the foot.

Often, developing one disorder that causes pain in the ball of the foot contributes to development of another disorder that causes pain in the same location.

Sesamoiditis is pain around a small bone (the sesamoid) below the metatarsal head where it adjoins the big toe (first metatarsal head). The cause of sesamoiditis is usually repeated injury. Sometimes the bone is fractured, or the bone or surrounding structures are inflamed. Sesamoiditis is particularly common among dancers, joggers, and those who have high-arched feet or wear high heels. The pain of sesamoiditis is felt beneath the ball of the foot at the big toe, is usually made worse by walking, and may be worse when wearing certain shoes. The area may be warm or swollen.

Plantar fasciitis is the most common condition of heel pain. This condition occurs when the long fibrous plantar fascia ligament along the bottom of the foot develops tears in the tissue resulting in pain and inflammation. The pain of plantar fasciitis is usually located close to where the fascia attaches to the calcaneous, also known as the heel bone. Plantar fasciitis causes the inflammation of the plantar fascia ligament that runs along the bottom of the foot. The plantar fascia ligament is made of fibrous bands of tissue and runs between the heel bone and the toes and stretches with every step. Inflammation develops when tears occur in the tissue.

The most common complaint from plantar fasciitis is a burning, stabbing, or aching pain in the heel of the foot. Most sufferers will be able to feel it in the morning because the fascia ligament tightens up during the night while we sleep, causing pain to diminish. In most cases, plantar fasciitis does not require surgery or invasive procedures to stop pain and reverse damage. Conservative treatments are usually all that is required. However, every person's body responds to plantar fasciitis treatment differently and recovery times may vary.

There are a number of plantar fasciitis causes. The plantar fascia ligament is like a rubber band and loosens and contracts with movement. It also absorbs significant weight and pressure. Because of this function, plantar fasciitis can easily occur from a number of reasons. Among the most common is an overload of physical activity or exercise. Athletes are particularly prone to plantar fasciitis and commonly suffer from it. Excessive running, jumping, or other activities can easily place repetitive or excessive stress on the tissue and lead to tears and inflammation, resulting in moderate to severe pain. Athletes who change or increase the difficulty of their exercise routines are also prone to overdoing it and causing damage.

A problem associated with treating the pain associated with the above-described conditions is the necessity to implant a pulse generator for providing the controls and power source for applying a stimulation signal to the appropriate nerve, such as the posterior tibial nerve. That is, for a relatively large implantable pulse generator, many patients do not have a calf of sufficient size to adequately serve as the implant location for the implantable pulse generator. As a result, the implantable pulse generator is positioned a significant distance from the electrode paddle, thereby requiring a lengthy wire that interconnects the implantable pulse generator to the electrode paddle. For example, the implantable pulse generator may be positioned in the patient's thigh or buttocks. The wire is therefore required to traverse the patient's knee, and is subject to bending and significant wear. This then leads to failure of the system causing one or more subsequent surgical events to become necessary to restore the system to working order. As a result, not only does the patient suffer when the system fails, but additional pain is endured because of the necessity for additional surgical procedures. In addition, higher health care costs are also incurred. Accordingly, there is a need for a system that address the shortcomings discussed above.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

A posterior tibial nerve stimulation system is provided for treating pain in the leg of a patient, such as pain in the foot of the patient. Thus, in at least one embodiment, a neurostimulation system is provided for treating pain in at least a portion of a leg of a patient, the system comprising:
an electrode comprising one or more contacts for transmitting an electrical signal to the leg of the patient;
a lead electrically connected to and extending from the electrode; and
an implantable pulse generator connected to the lead, the implantable pulse generator including at least one of:
(a) an arcuate shaped housing, and
(b) a shapeable housing comprising at least one adjustable structure, the at least one adjustable structure allowing the shapeable housing to be bent.

In at least one embodiment the neurostimulation assembly further comprises a wearable appliance for recharging the implantable pulse generator. In at least one embodiment the wearable appliance is selected from a group consisting of a sock, wrap, and sleeve. In at least one embodiment of the neurostimulation assembly the shapeable housing comprises a bendable metal structure. In at least one embodiment of the neurostimulation assembly the bendable metal structure comprises a plurality of metallic strands such as separated wires. In at least one embodiment of the neurostimulation assembly the bendable metal structure comprises a wire mesh. In at least one embodiment of the neurostimulation assembly the implantable pulse generator comprises an arcuate shaped battery. In at least one embodiment of the neurostimulation assembly the implantable pulse generator comprises a plurality of batteries. In at least one embodiment of the neurostimulation assembly at least two batteries of the plurality of batteries are separated from each other by a hinge. In at least one embodiment of the neurostimulation assembly the hinge comprises a locking mechanism. In at least one embodiment of the neurostimulation assembly the shapeable housing comprises at least one hinge. In at least one embodiment of the neurostimulation assembly the at least one hinge comprises a locking mechanism.

A nerve stimulation system is provided for treating pain in a limb of a patient, such as by way of example and not limitation, pain in the foot, ankle, knee, leg, hand, wrist, elbow, and/or arm of the patient. Thus, in at least one embodiment, a neurostimulation system is provided for treating pain in a limb of a patient, the system comprising:
an electrode including a contact for transmitting an electrical signal to the limb of the patient;
a lead electrically connected to and extending from the electrode; and
an implantable pulse generator connected to the lead, the implantable pulse generator including at least one of:
(a) an arcuate shaped housing, and
(b) a shapeable housing comprising at least one adjustable structure, the at least one adjustable structure allowing the shapeable housing to be bent.

The present invention encompasses a variety of possible configurations for the implantable pulse generator. Accordingly, a neurostimulation assembly for implanting in the calf region of a patient is provided, the neurostimulation assembly for treating pain in the foot of a patient, the neurostimulation assembly comprising:
an electrode comprising one or more contacts for transmitting an electrical signal to the leg of the patient;
a lead electrically connected to and extending from the electrode; and
an implantable pulse generator connected to the lead, the implantable pulse generator including a first node interconnected to a second node by an arcuate-shaped bridge, the arcuate-shaped bridge including a convex surface substantially matching a curvature of the calf region of the patient.

One or more embodiments described herein may be used to treat a variety of indications. Accordingly, a subcutaneously implantable neurostimulation system for treating pain in a limb of a patient is provided, the system comprising:
an electrode including a contact for transmitting an electrical signal to the limb of the patient;
a lead electrically connected to and extending from the electrode; and
an implantable pulse generator connected to the lead, the implantable pulse generator including an elongated housing member including one of a curved bar and an arcuate-shaped bridge, the elongated housing member connected to a first node, the first node including at least one of a battery and a control chip, wherein the elongated housing member comprises a height less than a height the first node.

In at least one embodiment of the subcutaneously implantable neurostimulation system the first node comprises at least one planar surface. In at least one embodiment of the subcutaneously implantable neurostimulation system the implantable pulse generator further comprises a second node, wherein the second node comprises a battery. In at least one embodiment of the subcutaneously implantable neurostimulation system the implantable pulse generator comprises a hinge. In at least one embodiment of the subcutaneously implantable neurostimulation system the implantable pulse generator comprises a bendable member.

Various components are referred to herein as “operably associated.” As used herein, “operably associated” refers to components that are linked together in operable fashion, and encompasses embodiments in which components are linked directly, as well as embodiments in which additional components are placed between the two linked components.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the present invention, a more particular description of the invention is rendered by reference to specific embodiments thereof which are illustrated in the appended drawings. It is appreciated that these drawings depict only typical embodiments of the invention and are therefore not to be considered limiting of its scope. The invention is described and explained with additional specificity and detail through the use of the accompanying drawings in which:

FIG. 1 is a perspective view of an embodiment of the present invention, wherein the apparatus is shown in an approximate implantation location in a patient's leg;

FIGS. 2a and 2b are perspective views of two appliances used to recharge a battery associated with the device illustrated in FIG. 1;

FIG. 3 is a perspective view of the implantable pulse generator shown in FIG. 1;

FIG. 4 is a top plan view of the device shown in FIG. 3;

FIG. 5a is a side elevation view of another embodiment of an implantable pulse generator;

FIG. 5b is a top plan view of the device shown in FIG. 5a after being deformed;

FIG. 5c is a top plan view of an embodiment of an implantable pulse generator;

FIG. 5d is a side elevation view of the device shown in FIG. 5c;

FIG. 5e is a top plan view of the device shown in FIGS. 5c and 5d after being deformed;

FIG. 6 is a side elevation view of an embodiment of an implantable pulse generator;

FIG. 7 is a side cutaway view of the device shown in FIG. 6;

FIG. 8 is a side elevation view of another embodiment of an implantable pulse generator;

FIG. 9 is a top plan view of the device shown in FIG. 8 after being deformed;

FIG. 10 is a perspective view of another embodiment of an implantable pulse generator;

FIG. 11 is a side cross-sectional view of the device shown in FIG. 10;

FIG. 12 is a back perspective view of another embodiment of an implantable pulse generator as well as a lead and an electrode paddle;

FIG. 13 is a back perspective view of yet another embodiment of an implantable pulse generator as well as a lead and an electrode paddle;

FIG. 14 is a top elevation view of the device shown in FIG. 13;

FIG. 15 is a rear elevation view of a patient's lower leg that includes the orientation of an embodiment when implanted;

FIG. 16 is a rear elevation view of a patient's lower leg that includes the orientation of an embodiment when implanted;

FIGS. 17-20 are possible orientations of implantable pulse generators relative to the patient's calf;

FIG. 21 is a front elevation view of a patient's leg with an implantable pulse generator located in the thigh;

FIG. 22 is a rear elevation view of a patient's leg with an implantable pulse generator located in the thigh;

FIG. 23 is a front elevation view of a patient's arm with an implantable pulse generator located in the forearm; and

FIG. 24 is a front elevation view of the stimulation system shown in FIG. 23.

The drawings are not necessarily to scale.

### DETAILED DESCRIPTION

Intractable pain in the plantar surface of the foot can be secondary to several conditions: plantar fasciitis, bunions, entrapped nerves, neuromas, nerve damage, diabetic neuropathy, to name a few. One or more embodiments of the one or more present inventions include an implantable pulse generator comprising one or more structural features for accommodating the shape of a portion of the patient's leg, such as the shape of the patient's calf. Embodiments may further include features for improving the esthetics of the implant after it is implanted within the patient.

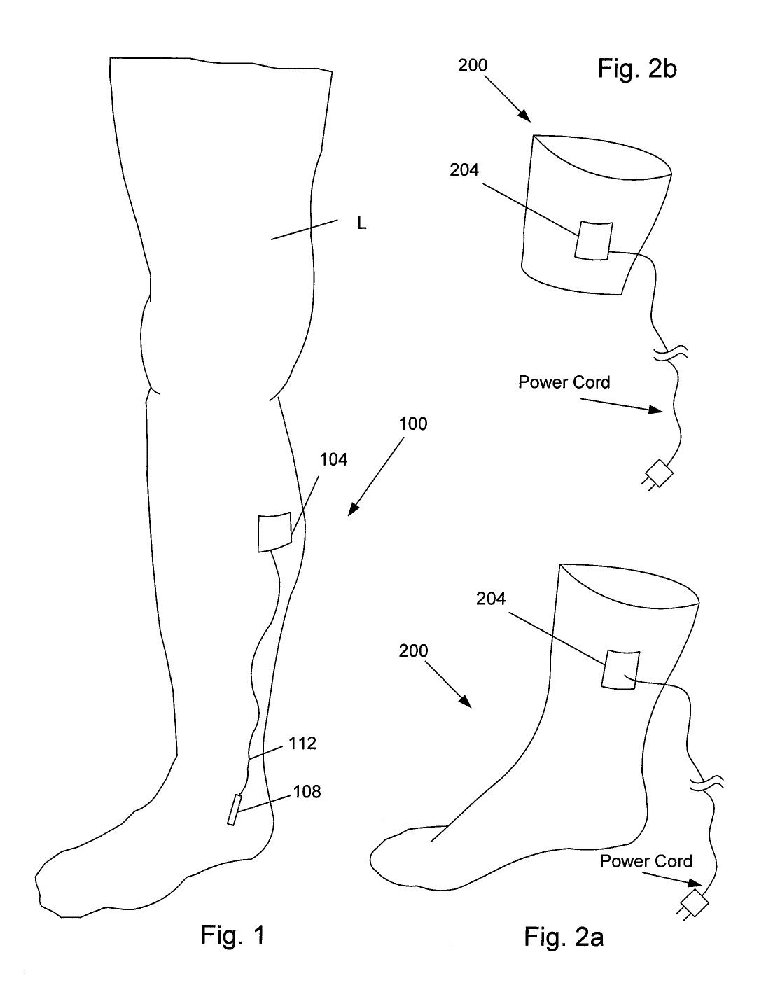

With reference now to FIG. 1, and in accordance with at least one embodiment, an implantable neurostimulation system 100 is shown in conjunction with a patient's leg L. The implantable neurostimulation system 100 includes an implantable pulse generator 104 connected to an electrode paddle 108 by lead 112. The electrode paddle 108 is positioned by the surgeon to reside in the vicinity of the patient's calf muscle, ankle region, or foot (as shown in FIG. 1). In at least one embodiment, the electrode paddle 108 is approximately 2.5 cm in length and approximately 1 cm in width. The electrode paddle 108 may comprise one or more electrodes or contacts, such as one or two columns of electrodes.

In at least one embodiment, the implantable neurostimulation system 100 (including the implantable pulse generator 104, lead 112, and electrode paddle 108), as well as the other systems shown herein, are surgically positioned to reside subcutaneously, that is, entirely under the patient's skin after the implantation procedure is completed.

In at least one embodiment, the implantable pulse generator 104 is relatively thin, such as about 0.5 to 1.0 cm in thickness. The implantable pulse generator 104 may comprise a rechargeable battery. In addition, in at least one embodiment the battery within the implantable pulse generator 104 is approximately the same shape as the exterior housing of the implantable pulse generator 104. That is, the battery may also comprise an arcuate shape. In at least one embodiment, the implantable pulse generator provides a maximum amperage output of about 10 mAmps, about PW 10 to 200 msec at a frequency or rate of about 10 to 100 Hz.

With reference now to FIGS. 2a and 2b, a recharging appliance 200 is provided in the form of a garment or attachable item, such as a sock/sock-type mesh (e.g., FIG. 2a) or sleeve/wrap (e.g., FIG. 2b) that can be worn by the patient. The recharging appliance 200 includes a recharging power source 204. In use, the recharging appliance 200 is situated such that the recharging power source 204 is in sufficiently close proximity to the implantable pulse generator 104 (that is implanted with the leg L of the patient) that the battery within the implantable pulse generator 104 can be recharged by a non-invasive system, such as an inductive link. Appropriate batteries are known to those skilled in the art, such as a nickel-metal hydride battery, or a lithium-ion or lithium-ion polymer rechargeable battery. Reference is made to U.S. Pat. No. 6,553,263 which is incorporated herein by reference.

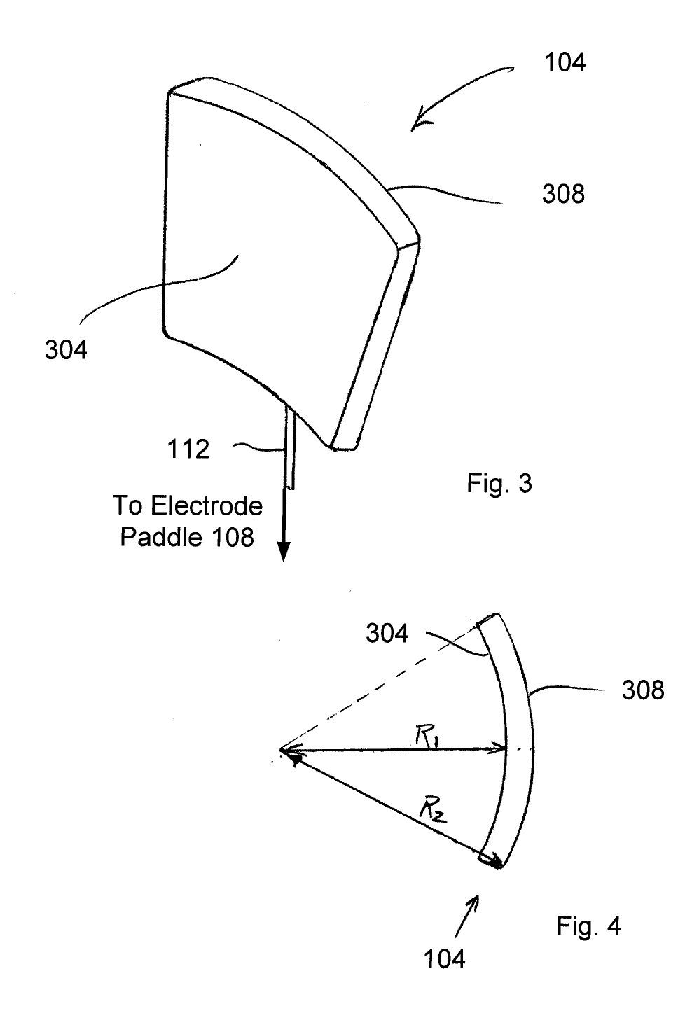

Referring now to FIG. 3, a detail perspective view of an embodiment of the implantable pulse generator 104 is shown. The implantable pulse generator 104 includes a first surface 304 and a second surface 308. The first surface 304 resides interior of the second surface 308 upon implanting within the patient's calf. The first surface 304 is concave and has a curvature for generally approximating the curvature of the patient's calf at the intended implant location. The second surface 308 is convex and resides closest to the patient's skin. The implantable pulse generator 104 may comprise a substantially trapezoid shape in rear elevation view, wherein the top comprises a greater width than the bottom. Accordingly, a variety of curvatures may be available to accommodate specific patient's needs, with smaller and greater curvatures to suite the size of the patient's calf.

Referring now to FIG. 4, in at least one embodiment, the radius of curvature R1 of the first surface 304 is smaller than the radius of curvature R2 of the second surface 308. In at least one embodiment, the first surface 304 may approximate an arc portion of a circle. Of course, other curved shapes are possible and are encompassed by this description and the claims herein.

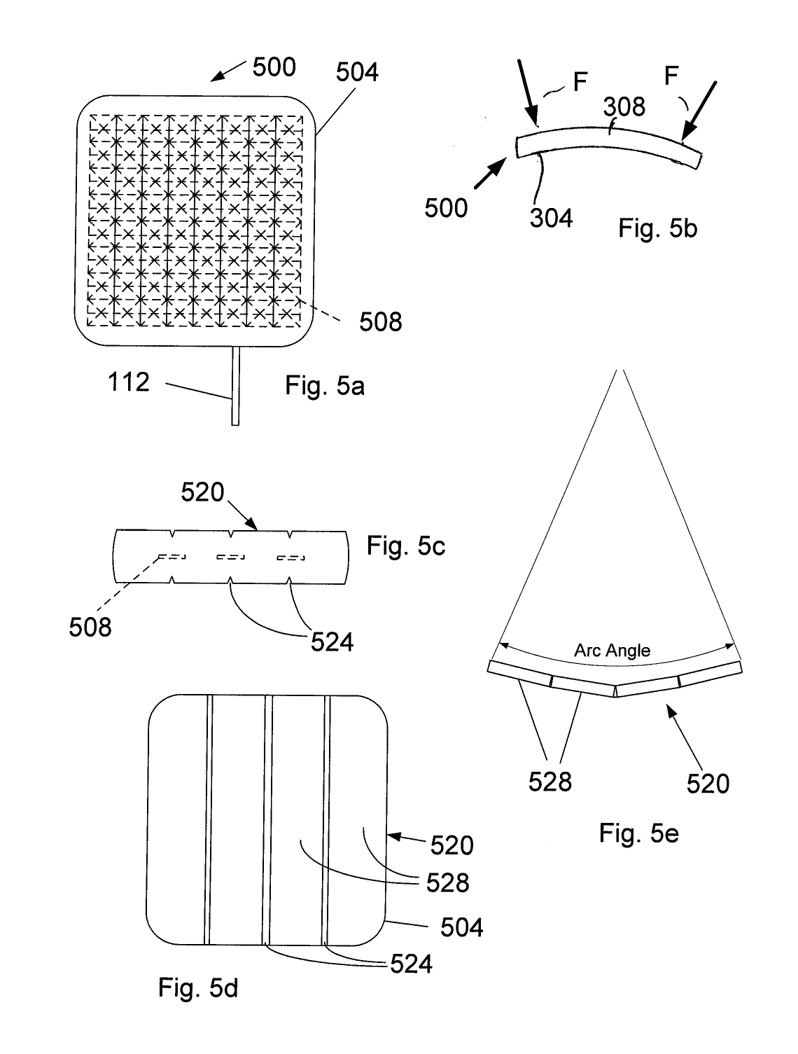

Referring now to FIG. 5a, a side elevation view of a shapeable implantable pulse generator 500 is shown. The deformable housing 504 of the shapeable implantable pulse generator 500 preferably comprises a flexible biocompatible material. The deformable housing 504 further includes a bendable structural material 508 that can be deformed to approximate the curvature of the patient's calf at the desired implant location. In at least one embodiment the bendable structural material 508 comprises a wire mesh. As seen in FIG. 5b, the shapeable implantable pulse generator 500 can be bent by applying a force F to the exterior of the deformable housing 504. For example, the surgeon can hold the shapeable implantable pulse generator 500 in his hands and apply a force F to shape the shapeable implantable pulse generator 500 as desired.

Referring now to FIG. 5c, a top elevation view of an implantable pulse generator 520 is shown that includes a plurality of groves 524 for allowing expansion and contraction of the surface material of the deformable housing 504 as the implantable pulse generator 520 is deformed to accommodate the patient's calf region. Distinct regions of bendable structural material 508 are provided in implantable pulse generator 520, and the distinct regions of the bendable structural material 508 are located within the interior area associated with the grooves 524. As seen in the side elevation view shown in FIG. 5d, panels 528 may be formed between the grooves 524, wherein the panels 528 may articulate relative to one another, but are generally non-bendable across the width of the panel 528. FIG. 5e illustrates the implantable pulse generator 520 with its panels 528. In at least one embodiment, the panels 528 are substantially non-bendable. Such non-bendable panels may accommodate one or more batteries and control chips for powering and controlling the function of the implantable pulse generator 520.

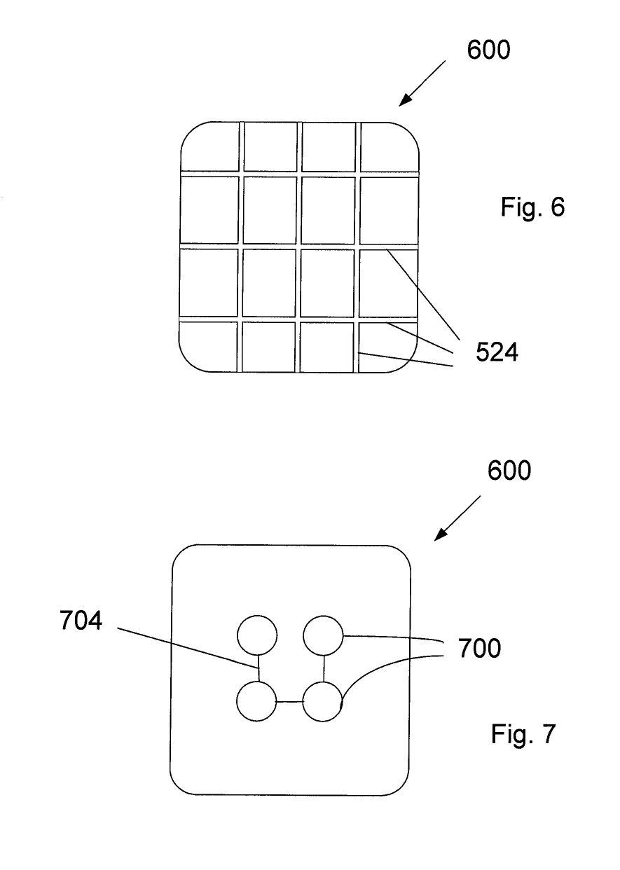

Referring now to FIG. 6, an implantable pulse generator 600 is shown that includes a plurality of grooves 524 oriented both in longitudinal (vertically relative to the page and a patient's calf) and latitudinal (horizontally relative to the page and a patient's calf) directions. Such a configuration allows the surgeon to accommodate the physical attributes of the shape of the patient's calf and shape the implantable pulse generator 600 both vertically and horizontally.

With reference now to FIG. 7, a cutaway view of the shapeable implantable pulse generator 600 is shown. In at least one embodiment, the shapeable implantable pulse generator 600 comprises a plurality of rechargeable batteries 700 that are electrically connected by wiring 704.

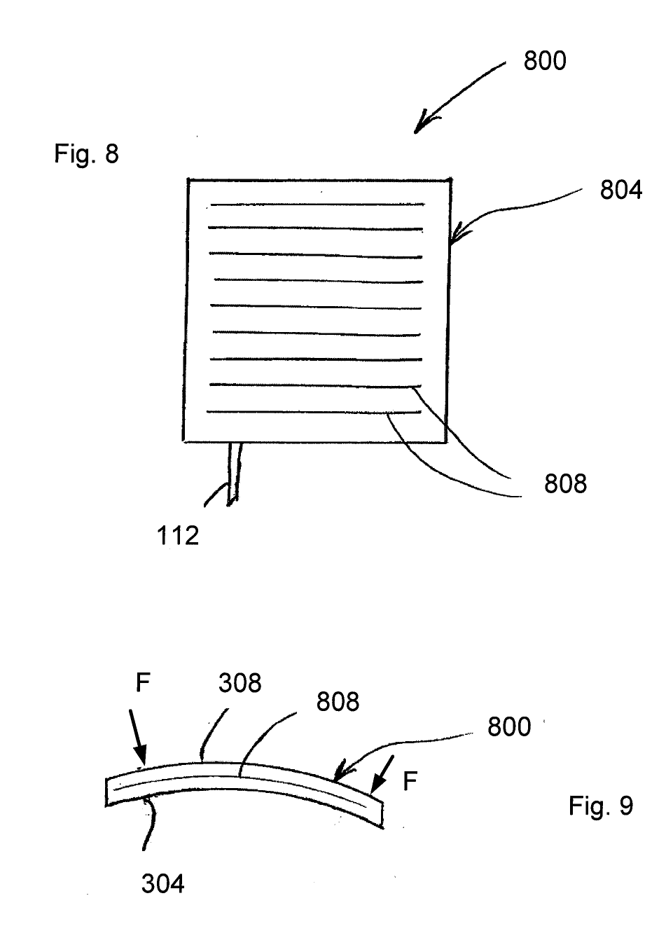

Referring now to FIGS. 8 and 9, another shapeable implantable pulse generator 800 is shown. The housing 804 includes a bendable structural material that can be curved to approximate the curvature of the patient's calf at the desired implant location. In at least one embodiment, the bendable structural material comprises a plurality of separated bendable structural wire strands 808. When bent under force F, the strands 808 maintain their curvature to approximate the shape of the patient's calf.

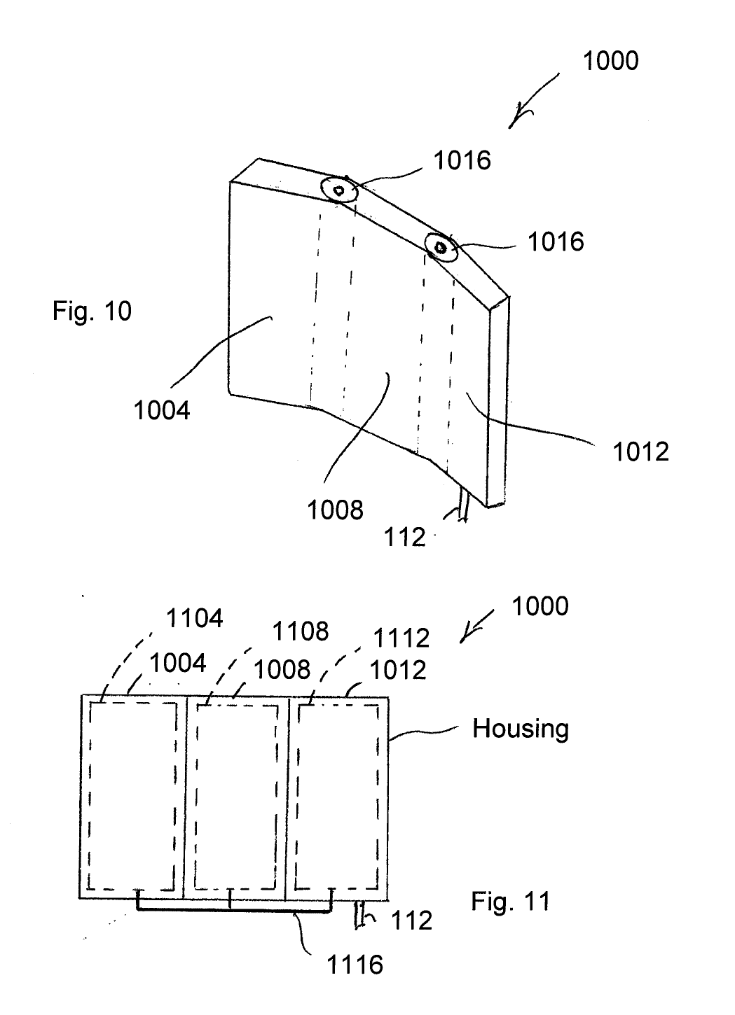

Referring now to FIG. 10, another embodiment of an implantable pulse generator 1000 is shown that includes a plurality of panel sections, such as panel sections 1004, 1008 and 1012. Panel section 1004 is interconnected to panel 1008 by a lockable hinge 1016. Similarly, panel section 1008 is interconnected to panel section 1012 by another lockable hinge 1016. The lockable hinges allow the surgeon to orient the panels to approximate the shape of the patient's calf at the location of implantation. The lockable hinge 1016 may comprise a screw residing within a hinge cylinder, wherein the screw can be tightened once the desired angle of the adjacent panels is set.

Referring now to FIG. 11, each of the panels includes a battery. That is, panel section 1004 includes battery 1104, panel section 1008 includes battery 1108, and panel section 1012 includes battery 1112. The batteries 1104, 1108 and 1112 are electrically connected by wiring 1116. Alternatively, panel section 1004 may comprise the control chip, while panel 1008 includes battery 1108, and panel section 1012 includes battery 1112. Thus, a variety of configurations are possible, with such range of configurations encompassed by the one or more present inventions.

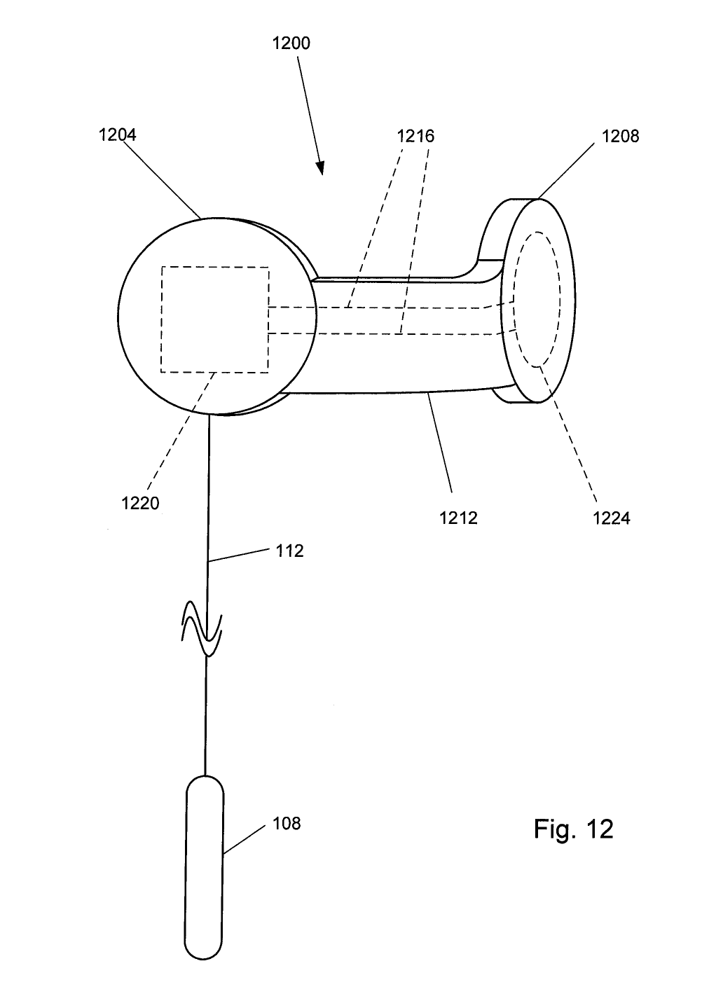

Referring now to FIG. 12, in another embodiment an implantable pulse generator 1200 includes a plurality of nodes. More particularly, for the implantable pulse generator 1200, a first node 1204 is spaced apart from a second node 1208, wherein the first node 1204 is interconnected to the second node 1208 by a curved or an arcuate-shaped bridge 1212 containing one or more conduits 1216, such as wires or conductive structures, for electrically connecting a device within the first node 1204 to a device in the second node 1208. It is noted that as used herein, “arcuate-shaped” is not meant to necessarily be limited to an arc portion of a circle, but rather, it is meant as a non-linear shape that substantially resembles the shape of a portion of the sides and back of the human limb to receive the device, such as a patient's forearm or calf. In at least one embodiment, the arcuate-shaped bridge 1212 spans the distance between the first node 1204 and the second node 1208, and serves as a conduit link between the first node 1204 and second node 1208 that are advantageously distributed around the leg of the patient so as not to require a relatively larger housing if both the control chip 1220 and battery 1224 were housed in a single-node device. Accordingly, one aspect of the implantable pulse generator 1200 is that a curved bridge, that is, the arcuate-shaped bridge 1212, has a smaller height than at least one of the first node 1204 and second node 1208, thereby minimizing the tissue displacement resulting from implanting the implantable pulse generator 1200 within the leg of a patient.

The implantable pulse generator 1200, therefore, includes features for reducing the profile of its various parts when implanted. The first node 1204 and second node 1208 are sized to accommodate one of either the control chip 1220 or battery 1224. Alternatively, depending upon the size of the control chip 1220 used, node 1204 or 1208 may include both a control chip 1220 and a battery 1224. The first node 1204 and second node 1208 serve to hold and protect the control chip 1220 associated with the implantable pulse generator 1200, and the arcuate-shaped bridge 1212 serves to distribute their location around a portion of the patient's leg while also serving as an electrical link.

In accordance with at least one embodiment, the arcuate-shaped bridge 1212 contains one or more batteries. By way of example and not limitation, at least a portion of a battery may reside within the arcuate-shaped bridge 1212, such that the battery itself is curved, and/or such that two or more relatively small batteries are used within the arcuate-shaped bridge 112.

In accordance with at least one embodiment, at least one node and/or the arcuate-shaped bridge 1212 may include a means for allowing the implantable pulse generator to be attached to the patient's internal tissue, such as tabs or apertures for receiving sutures, staples and/or similar anchoring materials.

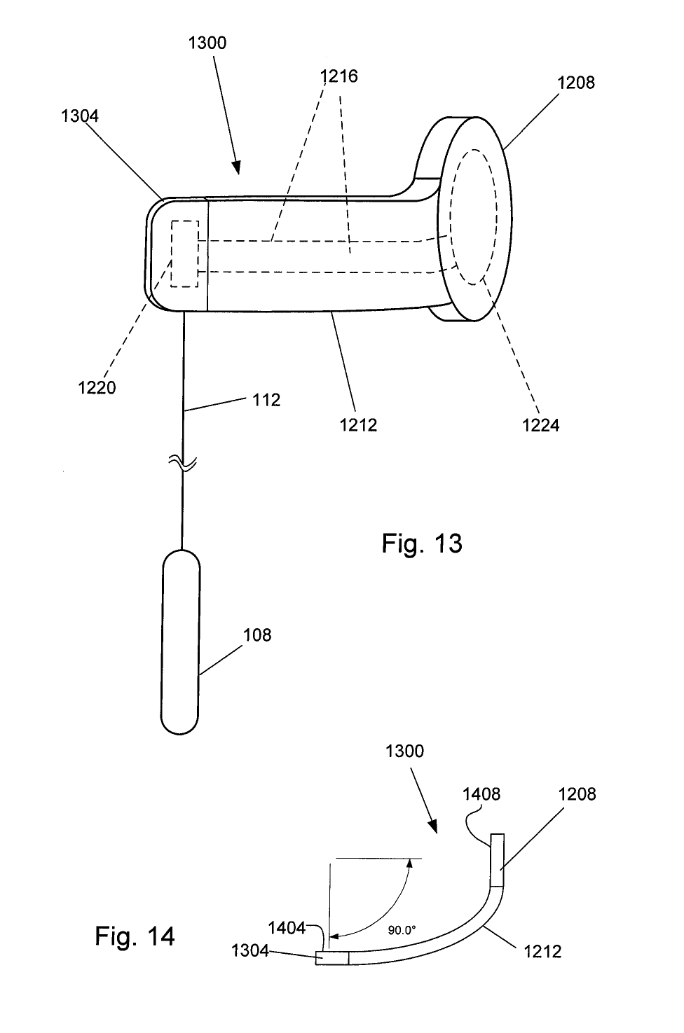

Referring now to FIG. 13, in another embodiment, an implantable pulse generator 1300 includes a first node 1304 connected to a second node 1208 by an arcuate-shaped bridge 1212, wherein the first node 1304 is a substantially flattened portion of the arcuate-shaped bridge 1212, and serves to house the control chip 1220. Those skilled in the art will appreciate that the conduits 1216 may be configured such that the lead 112 is connected to the first node 1304, second node 1208 or arcuate-shaped bridge 1212. Accordingly, FIG. 13 illustrates an example configuration, wherein the lead 112 is connected to the first node 1304.

In accordance with at least one embodiment, an implantable pulse generator substantially comprises a curved bar, wherein neither of the right or left ends (that is, the medial and lateral ends) have a size that differs substantially from the height of the arcuate-shaped bridge 1212 residing between the ends. In addition, the horizontal length of the curved bar, that is, its arc length, is greater than a vertical height of the curved bar (as oriented when implanted). Thus, the curved bar is implanted below the patient's skin and it fits around the patient's leg, thereby distributing its profile to be similar to the lateral curvature of the patient's calf. Here, it is noted that in at least one embodiment, the curved bar is made of a substantially non-bendable material. Alternatively, at least portions of the curved bar may comprise bendable elements, such as those described above, that permit the surgeon to customize the fit of the implantable pulse generator.

Referring now to FIG. 14, a plan view of an exemplary version of the implantable pulse generator 1300 is shown that depicts the orientation of the first node 1304 relative to the second node 1208 and the arcuate-shaped bridge 1212 that interconnects the first node 1304 to the second node 1208. For the example configuration shown in FIG. 14, an inside wall surface 1404 of the first node 1304 is substantially perpendicular to an inside wall surface 1408 of the second node 1208. It is to be understood that the configuration of the first node 1304 relative to the second node 1208 may differ from that shown in FIG. 14, and that FIG. 14 illustrates but one example. The orientation shown in FIG. 14 also applies to other embodiments disclosed herein, including the implantable pulse generator 1200 shown in FIG. 12.

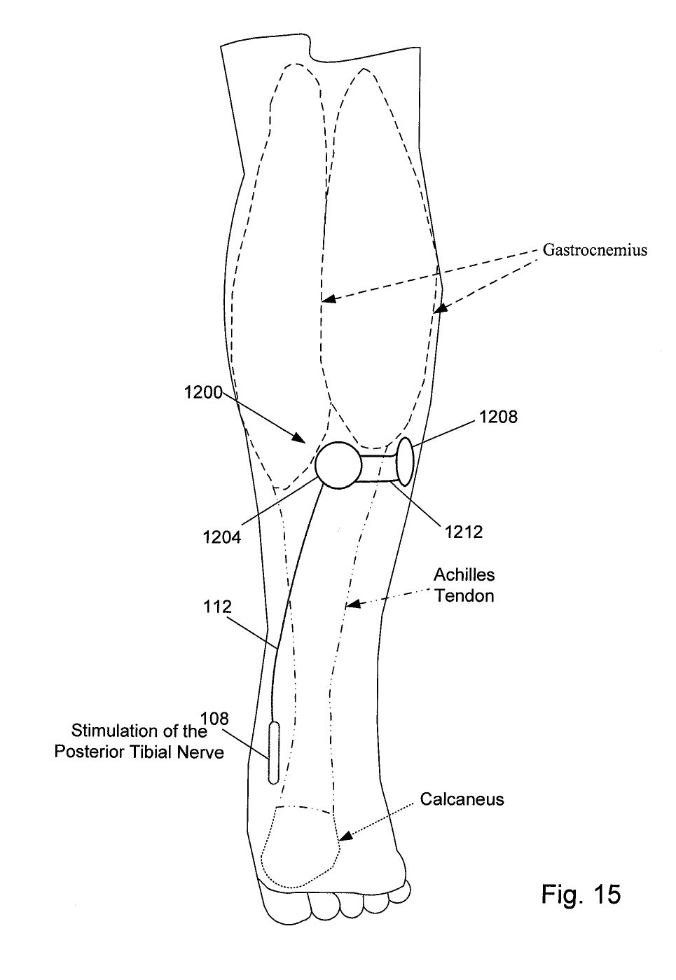

With reference now to FIG. 15, the implantable pulse generator 1200 is shown in one possible configuration when implanted to provide stimulation via the electrode paddle 108 to the posterior tibial nerve. As illustrated in FIG. 15, the first node 1204 of the implantable pulse generator 1200 is located near the base of the gastrocnemius muscles of the calf. In addition, a second node 1208 of the implantable pulse generator 1200 is configured to be located inferior to the base of the lateral gastrocnemius muscle. Accordingly, in at least one embodiment, and by way of example and not to be limiting, the first node 1204 is situated substantially on the posterior side or at the back of the leg (that is, about 180 degrees from the direction of the patient's forward facing toes), while the second node 1208 is situated approximately 60 to 90 degrees away from the first node 1204 and to the lateral (or outer) side of the patient's leg.

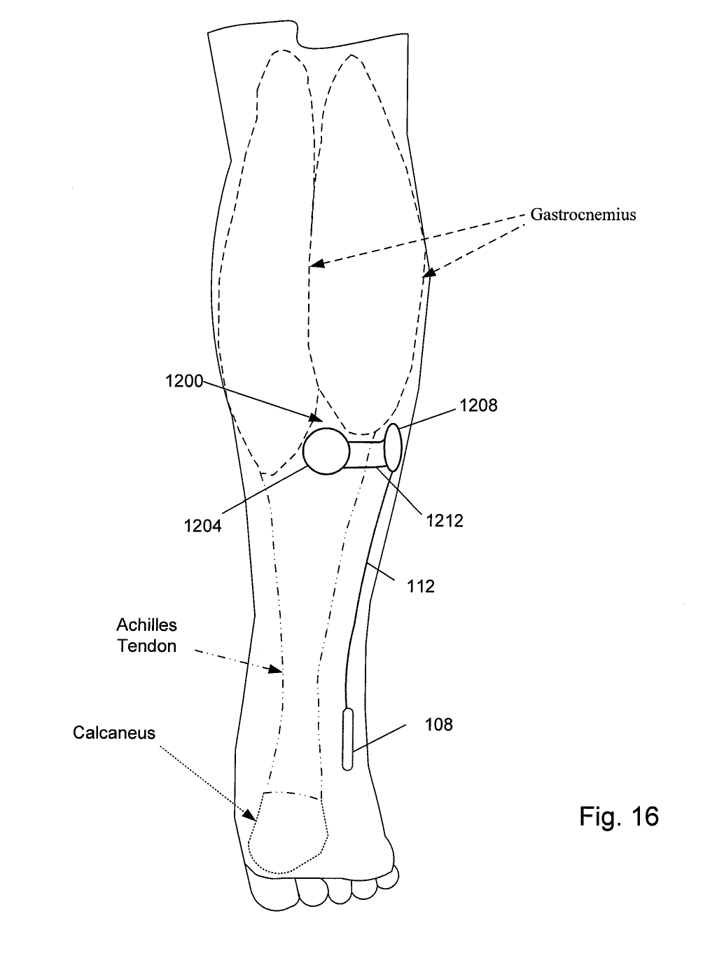

With reference now to FIG. 16, the implantable pulse generator 1200 is shown in a configuration when implanted similar to that illustrated in FIG. 15, but with the lead 112 and the electrode paddle 108 oriented for stimulation of different portions of the anatomy from that of the posterior tibial nerve. Accordingly, the different embodiments described herein have a variety of uses and implant locations.

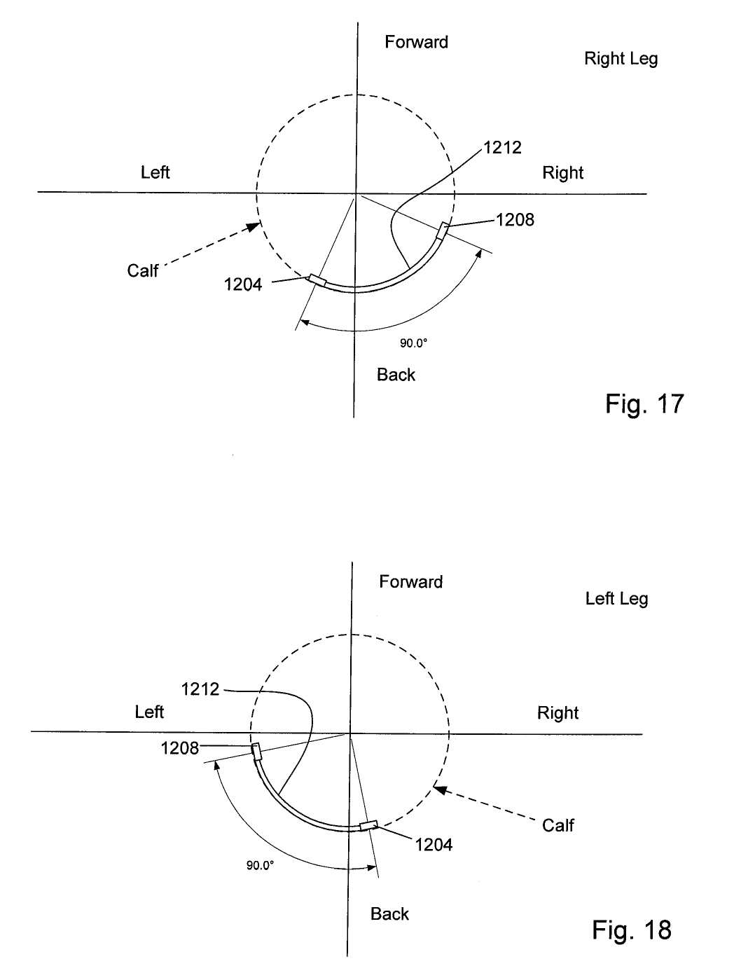

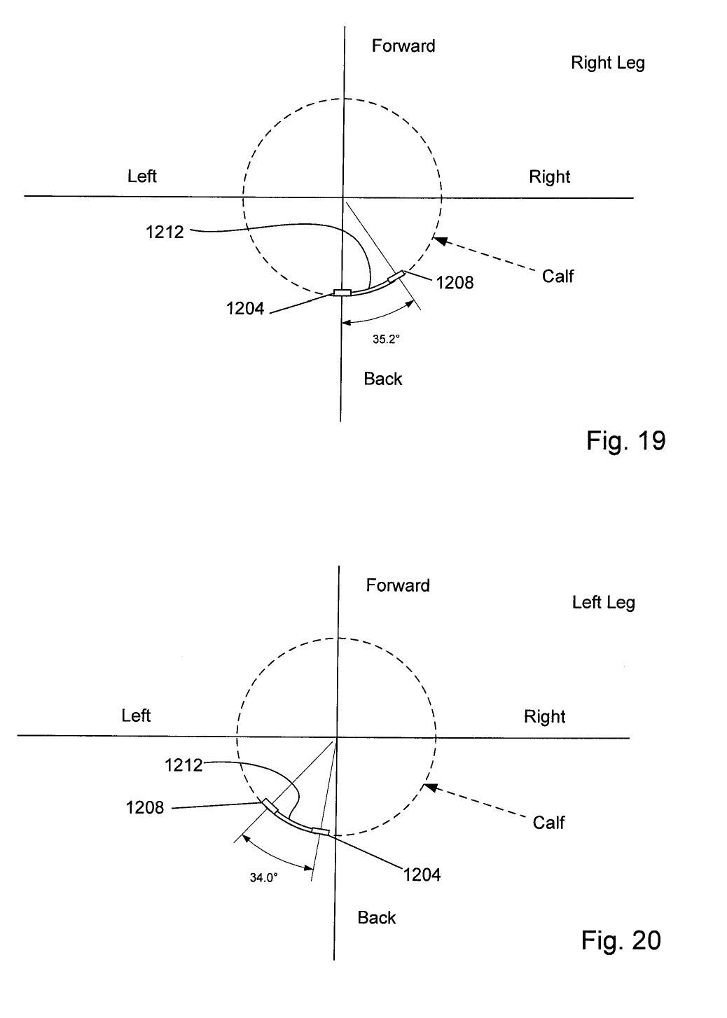

With reference now to FIG. 17, one possible configuration is illustrated for the positioning of the first node 1204 and second node 1208 when implanting in the right leg, where the nodes 1204 and 1208 are separated by about a 90 degree angle. A similar configuration is shown in FIG. 18 for the left leg. In FIG. 19, another possible configuration is shown for the right leg wherein the first node 1204 is separated from the second node by an angle of approximately 35 degrees. FIG. 20 illustrates a similar configuration for the left leg. Accordingly, the location of the nodes 1204 and 1208 can vary, as can the angle separating the nodes 1204 and 1208.

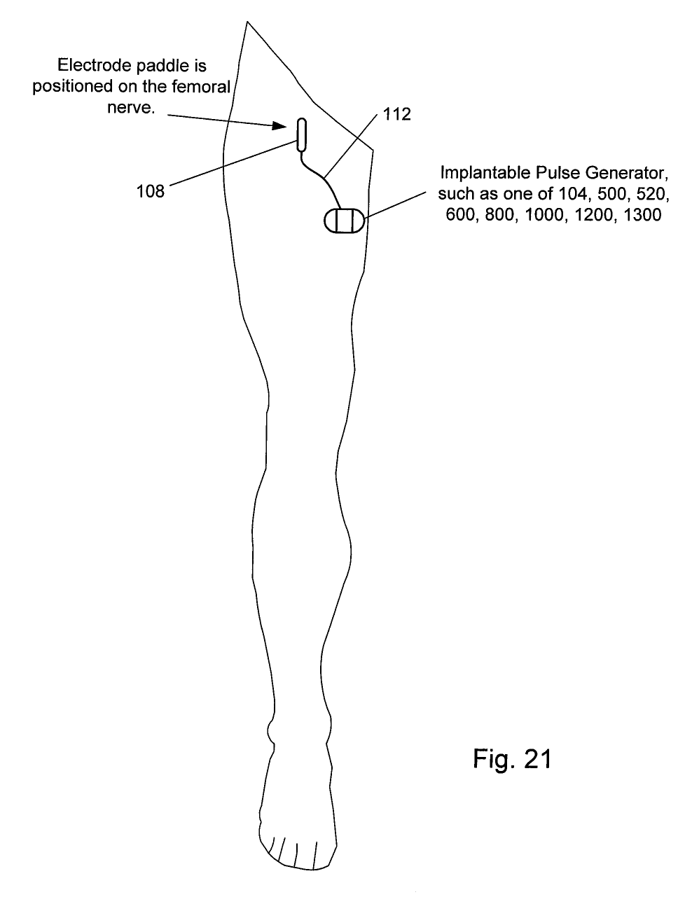

Referring now to FIG. 21, and in accordance with at least one embodiment, a locally positioned implantable pulse generator is positioned in a patient's thigh for providing energy to an electrode positioned for stimulation of nerves also located within the patient's leg. More particularly, an implantable pulse generator, such as one of 104, 500, 520, 600, 800, 1000, 1200, or 1300 is shown after being implanted in the patient's anterior thigh. The implantable pulse generator is connected to lead 112, which provides an electrical signal to electrode paddle 108, wherein the lead 112, electrode paddle 108 and implantable pulse generator are all implanted under the patient's skin. As shown in the example placement depicted in FIG. 21, the electrode paddle 108 is positioned to provide stimulation to the femoral nerve.

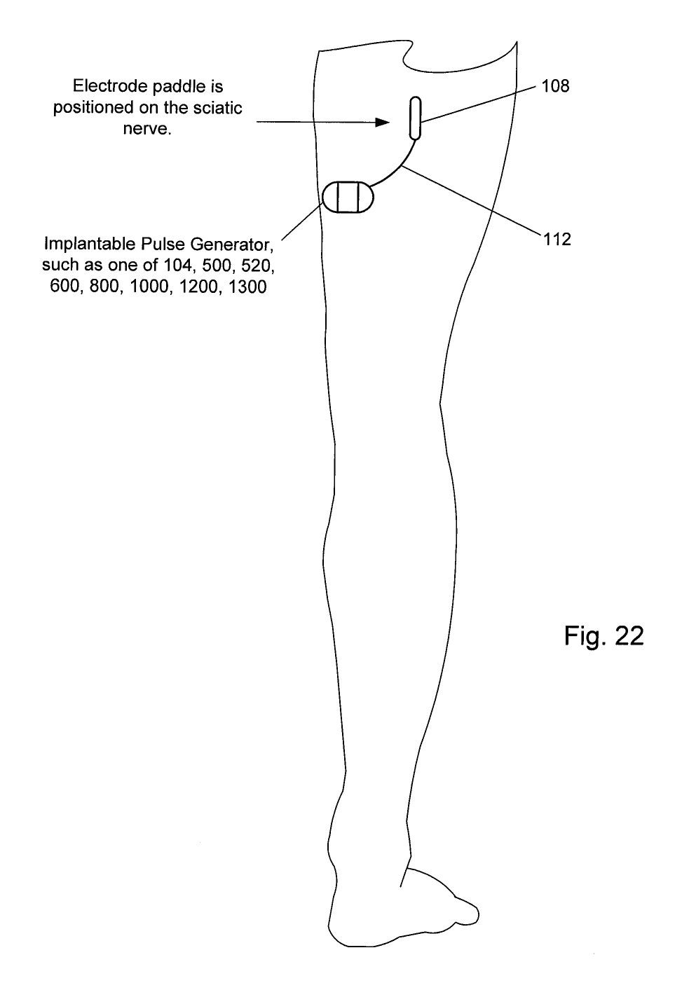

Referring now to FIG. 22, and in accordance with at least one embodiment, a locally positioned implantable pulse generator is positioned in a patient's posterior thigh for providing energy to an electrode positioned for stimulation of nerves also located within the patient's leg. More particularly, an implantable pulse generator, such as one of 104, 500, 520, 600, 800, 1000, 1200, or 1300 is shown after being implanted in the patient's posterior thigh. The implantable pulse generator is connected to lead 112, which provides an electrical signal to electrode paddle 108, wherein the lead 112, electrode paddle 108 and implantable pulse generator are all implanted under the patient's skin. As shown in the example placement depicted in FIG. 22, the electrode paddle 108 is positioned to provide stimulation to the sciatic nerve.

Based on the illustrations shown in FIGS. 21 and 22, as well as the associated description, it will be appreciated by those skilled in the art that alternative locations and/or multiple locations for electrode placement may be used in the patient's leg as may be indicated for the treatment of the patient's pain. Use of a neurostimulation system consistent with those described herein may be appropriate. Accordingly, the different embodiments described herein and their modified versions have a variety of uses and implant locations, including in the patient's legs.

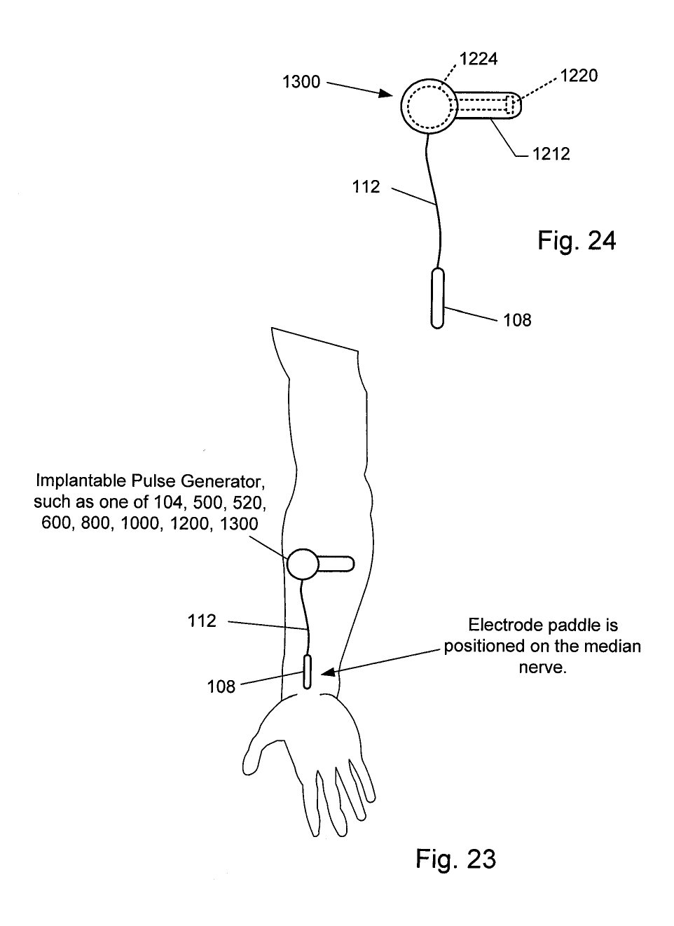

Referring now to FIG. 23, and in accordance with at least one embodiment, a locally positioned implantable pulse generator is positioned in a patient's arm for providing energy to an electrode positioned for stimulation of nerves also located within the patient's arm. More particularly, an implantable pulse generator, such as one of 104, 500, 520, 600, 800, 1000, 1200, or 1300 is shown after being implanted in the patient's forearm. The implantable pulse generator is connected to lead 112, which provides an electrical signal to electrode paddle 108, wherein the lead 112, electrode paddle 108 and implantable pulse generator are all implanted under the patient's skin. As shown in the example placement depicted in FIG. 23, the electrode paddle 108 is positioned to provide stimulation to the median nerve. It will be appreciated by those skilled in the art that alternative locations and/or multiple locations for electrode placement may be used in the patient's arm as may be indicated for the treatment of the patient's pain. Use of a neurostimulation system consistent with those described herein may be appropriate. Accordingly, the different embodiments described herein and their modified versions have a variety of uses and implant locations, including in the patient's arms.

By way of example and not limitation, FIG. 24 illustrates an embodiment of an implantable pulse generator 1300 for use in the patient's arm. The system includes an arcuate-shaped bridge 1212; however, the curvature of the arcuate-shaped bridge 1212 used in the forearm of a patient may vary in curvature from that used in the calf region. Accordingly, embodiments described herein include variations of the devices described and shown, and such variations are encompassed by this description and the claims herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The one or more present inventions, in various embodiments, include components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention (e.g., as may be within the skill and knowledge of those in the art, after understanding the present disclosure). It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A neurostimulation assembly for treating pain in at least a portion of a limb of a patient, comprising:
an electrode comprising one or more contacts for transmitting an electrical signal to the limb of the patient;
a lead electrically connected to and extending from the electrode; and
an implantable pulse generator connected to the lead, the implantable pulse generator including at least one of:
(a) an arcuate-shaped housing, and
(b) a shapeable housing comprising at least one adjustable structure, the at least one adjustable structure allowing the shapeable housing to be bent.

2. The neurostimulation assembly of claim 1, further comprising a wearable appliance for recharging the implantable pulse generator.

3. The neurostimulation assembly of claim 2, wherein the wearable appliance is selected from a group consisting of a sock, wrap, and sleeve.

4. The neurostimulation assembly of claim 1, wherein the shapeable housing comprises a bendable metal structure.

5. The neurostimulation assembly of claim 4, wherein the bendable metal structure comprises a plurality of separated wires.

6. The neurostimulation assembly of claim 4, wherein the bendable metal structure comprises a wire mesh.

7. The neurostimulation assembly of claim 1, wherein the implantable pulse generator comprises an arcuate-shaped battery.

8. The neurostimulation assembly of claim 1, wherein the implantable pulse generator comprises a plurality of batteries.

9. The neurostimulation assembly of claim 8, wherein at least one battery of the plurality of batteries are separated from another battery of the plurality of batteries by a hinge.

10. The neurostimulation assembly of claim 9, wherein the hinge comprises a locking mechanism.

11. The neurostimulation assembly of claim 1, wherein the shapeable housing comprises at least one hinge.

12. The neurostimulation assembly of claim 11, wherein the at least one hinge comprises a locking mechanism.

13. A neurostimulation assembly for implanting in a calf region of a patient, the neurostimulation assembly for treating pain in a foot of a patient, the neurostimulation assembly comprising:
an electrode comprising one or more contacts for transmitting an electrical signal to the patient;
a lead electrically connected to and extending from the electrode; and
an implantable pulse generator connected to the lead, the implantable pulse generator including a first node interconnected to a second node by an arcuate-shaped bridge, the arcuate-shaped bridge including a convex surface substantially matching a curvature of the calf region of the patient.

14. The neurostimulation assembly of claim 13, wherein the arcuate-shaped bridge comprises a height less than a height of at least one of the first node and the second node.

15. The neurostimulation assembly of claim 13, wherein the first node comprises a control chip and the second node comprises a rechargeable battery.

16. A subcutaneously implantable neurostimulation system for treating pain in a limb of a patient, the subcutaneously implantable neurostimulation system comprising:
an electrode including a contact for transmitting an electrical signal to the limb of the patient;
a lead electrically connected to and extending from the electrode; and
an implantable pulse generator connected to the lead, the implantable pulse generator including an elongated housing member including one of a curved bar and an arcuate-shaped bridge, the elongated housing member connected to a first node, the first node including at least one of a battery and a control chip, wherein the elongated housing member comprises a height less than a height the first node.

17. The subcutaneously implantable neurostimulation system of claim 16, wherein the first node comprises at least one planar surface.

18. The subcutaneously implantable neurostimulation system of claim 16, further comprising a second node, wherein the second node comprises a battery.

19. The subcutaneously implantable neurostimulation system of claim 16, wherein the implantable pulse generator comprises a hinge.

20. The subcutaneously implantable neurostimulation system of claim 16, further wherein the implantable pulse generator comprises a bendable member.
